(19) United States
(12) Patent Application Publication
(10) Pub. No.: US 2012/0064558 A1
(43) Pub. Date: Mar. 15, 2012

(54) Assays for Differentiating between Ischemic and Hemorrhagic Stroke in a Human Patient

(76) Inventors: Mary Frances LOPEZ, Boston, MA (US); Amol PRAKASH, Cambridge, MA (US); David A. SARRACINO, Andover, MA (US); Taha REZAI, Cambridge, MA (US); Bryan E. KRASTINS, Somerville, MA (US); Michael J. ATHANAS, Wayland, MA (US)

(21) Appl. No.: 12/881,115

(22) Filed: Sep. 13, 2010

(51) Int. Cl. C12Q 1/37

(52) U.S. Cl. 435/23

(57) ABSTRACT

Methods are disclosed for rapidly and reliably differentiating between ischemic and hemorrhagic stroke events in a human patient. The methods include obtaining a biological sample, such as blood serum, and analyzing the sample or a derivative thereof by mass spectrometry to measure the amount of one or more targeted proteins differentially present and ischemic and hemorrhagic stroke patients. The analysis may include proteolytic digestion of the biological sample and selective reaction monitoring (SRM) tandem mass spectrometry of the digested sample to determine the amounts of surrogate peptides corresponding to the targeted proteins.

### REFERENCE TO A SEQUENCE LISTING SUBMITTED AS A TEXT FILE VIA EFS-WEB

The official copy of the sequence listing is submitted concurrently with the specification as a text file via EFS-Web, in compliance with the American Standard Code for Information Interchange (ASCII), with a file name of 5448US1-NAT_SequenceListing_ST25.txt, a creation date of Sep. 13, 2010, and a size of 2 KB. The sequence listing filed via EFS-Web is part of the specification and is hereby incorporated herein in its entirety by reference.

### FIELD OF THE INVENTION

The present invention relates generally to methods for the diagnosis of stroke in a human patient, and more particularly to a method of differentiating between ischemic and hemorrhagic stroke by mass spectrometric analysis of a biological sample.

### BACKGROUND OF THE INVENTION

A stroke, or cerebrovascular accident, is the rapidly developing loss of brain function due to disturbance of blood supply to the brain. Strokes can be classified into two major types: ischemic and hemorrhagic. Ischemic strokes occur due to decreased blood flow arising from the formation of a clot or other obstruction. Hemorrhagic strokes, which constitute about thirteen percent of total strokes but are responsible for approximately thirty to forty percent of stroke-related deaths, are caused by rupture of a blood vessel or an abnormal vascular structure.

Proper medical treatment of a stroke victim relies on accurate differentiation between ischemic and hemorrhagic stroke. For example, ischemic stroke is typically treated by the administration of tissue plasminogen activator (tPA) or other “clot busting” agent designed to dissolve clots, thereby restoring blood flow to the brain. However, if tPA or a similar drug is given to a patient experiencing a hemorrhagic stroke, intracerebral bleeding will be worsened, thereby increasing the severity of the stroke and its consequent damage. In current practice, diagnosis of stroke type is performed by inspection of images obtained by computerized tomography (CT) or magnetic resonance imaging (MRI) scans to ascertain the presence of bleeding in or around the patient's brain. While this technique has been shown to be highly selective if skillfully employed, it requires the availability of expensive and complex CT or MRI equipment, and may be subject to error or uncertainty if the medical personnel conducting and/or interpreting the scan are inexperienced or inadequately trained. Furthermore, the acquisition of a CT or MRI scan may be time consuming and thereby delay the administration of appropriate treatment to the patient.

Against this background, there remains a need in the art for a reliable, rapid, and relatively inexpensive method for differentiating between ischemic and hemorrhagic stroke in a human patient.

### SUMMARY

Embodiments of the present invention arise from the discovery that certain proteins, referred to as biomarkers, are present in the blood of ischemic stroke patients in different quantities relative to hemorrhagic stroke patients. In accordance with an illustrative embodiment, a method of differentiating between ischemic and hemorrhagic stroke in a human patient includes obtaining a biological sample, such as a blood serum sample, from the patient, and then analyzing the sample by mass spectrometry to measure the amount of at least one targeted protein selected from the group consisting of apolipoprotein A-I preprotein, apolipoprotein A-II preprotein, apolipoprotein A-IV preprotein, apolipoprotein B precursor, apolipoprotein C-I precursor, apolipoprotein C-II precursor, apolipoprotein C-III precursor, apolipoprotein D precursor, apolipoprotein E precursor and apolipoprotein H precursor. The measured amount(s) is or are compared to a predetermined control amount, and the results of this comparison provide an indication as to whether the patient has experienced an ischemic or hemorrhagic stroke.

In a more specific implementation, a blood serum sample is subjected to enzymatic digestion prior to mass spectrometric analysis to produce peptide fragments from the targeted protein(s). The digested sample, or a purified fraction thereof, is subjected to tandem mass spectrometry to measure the amount of one or more peptide fragments produced by and characteristic of the targeted protein(s). The tandem mass spectrometry analysis may be performed in a triple quadrupole mass spectrometer operated in selective reaction monitoring mode (SRM), whereby one or more precursor-to-product ion transitions specific to the peptide fragments are monitored. Quantitation of the surrogate peptide fragments may be facilitated by the addition of internal standards to the sample (either prior or subsequent to digestion), which may take the form of synthetic isotopically-labeled versions of the peptide fragments. An assay for differentiation of ischemic and hemorrhagic stroke may be based on measurement of a single biomarker protein or on measurement of multiple biomarker proteins (known as a biomarker panel), including the determination of the relative amounts of two or more biomarker proteins.

Mass spectrometric assays of the type described herein advantageously provide the ability to rapidly and confidently discriminate between ischemic and hemorrhagic stroke based on the differential presence of one or more biomarkers in a biological sample, while avoiding the need for expensive, time-consuming and potentially difficult to interpret brain imaging techniques such as CT and MRI scans.

### BRIEF DESCRIPTION OF THE DRAWINGS

In the accompanying drawings:

FIG. 1 is a flowchart illustrating the steps of a method for differentiating between ischemic and hemorrhagic stroke by mass spectrometric analysis of a biological sample, in accordance with an illustrative embodiment of the invention; and

FIG. 2 is a receiver operatic characteristic (ROC) curve showing the experimentally determined sensitivity-selectivity relationship for the differential determination of hemorrhagic versus ischemic stroke by mass spectrometry-based measurement of the amount of apolipoprotein A-IV preprotein in blood serum samples.

### DETAILED DESCRIPTION OF EMBODIMENTS

Embodiments of the present invention generally include steps of analyzing a biological sample by mass spectrometry to measure the amount of one or more targeted proteins, and comparing the measured amount(s) to corresponding control amount(s). As used herein, measuring the amount of a targeted protein is deemed to include any technique or combination of techniques by which the relative or absolute quantity of the targeted protein and/or its surrogate peptide(s) may be inferred. The amount of the targeted peptide may be measured indirectly, as discussed below, by enzymatically digesting the targeted protein to produce characteristic peptide fragments prior to mass analysis, and measuring the amount of the one or more characteristic peptide fragments, either as an absolute quantity or a relative quantity referenced to the amount of another peptide fragment.

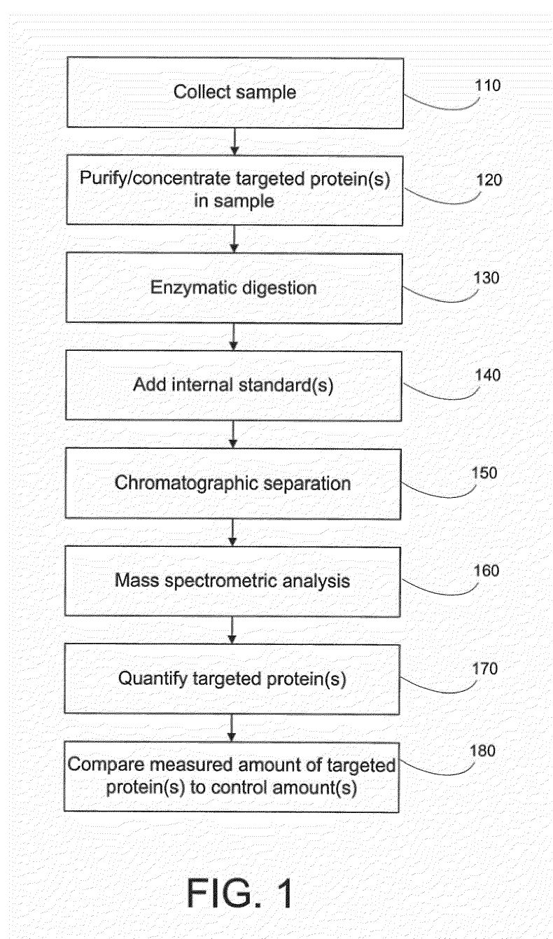

Referring initially to FIG. 1, which depicts the steps of a mass spectrometry-based assay for differentiating between ischemic and hemorrhagic stroke in a human patient, a biological sample is collected from the patient using a standard protocol, step 110. Preferably, the biological sample is a blood serum sample obtained by venipuncture with subsequent fractionation to remove cells and clotting factors. In alternative embodiments, the sample may take the form of another biofluid such as blood plasma, sweat, saliva, urine or lymphatic fluid, or a tissue lysate.

The sample is then subjected to a series of preparation steps to preserve, concentrate and purify the targeted protein(s) to be measured by mass spectrometry in order to improve the overall sensitivity of the assay, step 120. Such preparation steps are well known in the art and may include, for example, any one or combination of immunoaffinity capture, size exclusion chromatography, affinity chromatography, sequential extraction and gel electrophoresis.

Certain preferred embodiments of the invention will utilize a “bottom up” approach, whereby the sample is enzymatically digested prior to mass spectrometric analysis, step 130. Digestion of the targeted protein with a proteolytic enzyme such as trypsin produces, by cleaving of selected amino acid bonds, a set of peptide fragments, one or more of which is characteristic of the associated protein. Such fragments are referred to as surrogate or fingerprint peptide fragments, and the measured amount of the surrogate peptide fragment(s) serves as a proxy for the amount of the targeted protein in the sample. Proteolytic fragmentation is particularly useful for high molecular weight target proteins because their surrogate peptide fragments are more easily monitored and resolved by mass spectrometry. While the FIG. 1 flowchart depicts the digestion step as sequentially following preparation steps 120, proteolytic digestion of the sample may be performed prior to all or some of the preparation steps. In other implementations of the assay, the target protein(s) or the surrogate peptide fragment(s) may be chemically modified to improve the sensitivity of the mass spectrometric analysis (e.g., by promoting better ionization) or to assist in distinguishing the peptide fragment ions from isobaric interferences in the resulting spectrum.

To facilitate accurate quantitation of the surrogate peptides by mass spectrometry, a set of isotopically-labeled synthetic versions of the surrogate peptides may be added in known amounts to the sample for use as internal standards, step 140. Since the isotopically-labeled peptides have physical and chemical properties substantially identical to the corresponding surrogate peptide, they co-elute from the chromatographic column and are easily identifiable on the resultant mass spectrum. The internal standards may be added to the sample during the sample prior to completion of the preparation or digestion steps.

Next, the purified and digested sample containing the surrogate peptides (and associated internal standards) is chromatographically separated by using a suitable HPLC column and elution protocol in order to temporally isolate different compounds in the prepared sample and simplify the process of identifying and quantifying substances in the mass spectra, step 150. In a preferred mode of the invention, chromatographic separation is performed on-line, such that the column eluate is directly introduced into the inlet of the ion source of the mass spectrometer, for example through an electrospray ionization probe. Alternatively, fractions of the eluate may be collected and stored for later mass analysis. Other suitable separation techniques known in the art, such as electrophoresis, may be utilized in place of or in addition to HPLC.

In step 160, the prepared sample is ionized, for example by electrospray ionization, and the resultant ions are analyzed by mass spectrometry to detect and quantify a set of surrogate peptide fragments present in the sample. In the present example, mass spectrometric analysis of the prepared sample may be carried out in any instrument capable of monitoring multiple transitions in SRM mode. In a preferred implementation, the prepared sample is analyzed in a triple quadrupole mass spectrometer, such as the Thermo Scientific Vantage TSQ instrument. Triple quadrupole mass spectrometers utilize two resolving quadrupoles, also referred to as quadrupole mass filters, each of which is operable to selectively transmit only ions having a mass-to-charge ratio (m/z) of interest. An RF-only quadrupole filled with a collision gas (a collision cell) is interposed in the ion path between the two resolving quadrupoles. Precursor ions selected in the first resolving quadrupole undergo energetic collisions in the collision cell to yield fragment ions. The fragment ions pass into the second resolving quadrupole, and the selectively transmitted product ions strike a detector, which generates a signal representative of the quantity of transmitted ions. A specified pair of precursor/product ion m/z's to which the first and second resolving quadrupoles are tuned is referred to as a transition. The instrument controller, which controls the RF and resolving DC voltages individually applied to the first and second resolving quadrupoles, is configured to rapidly cycle among individual transitions in a stored list such that a large number of different transitions may be concurrently monitored. The number of transitions that may be concurrently monitored in a given time period will depend on various operational factors, including the settling time of the resolving quadrupoles and the rate at which product ions are removed from the collision cell; commercially available instruments are typically capable of monitoring a few hundred transitions per second.

Due to the multiplicity of charge states and fragmentation pathways that exist for each peptide fragment, monitoring all possible precursor-to-product ion transitions for the group of surrogate peptide fragments is generally impractical. Instead, the mass spectrometer is operated to monitor a subset of transitions selected to optimize sensitivity and selectivity for each surrogate peptide fragment. In a typical assay, a set of 4-8 transitions are monitored for each targeted peptide fragment, comprising 2-4 transitions for the wild-type peptide (i.e., the peptide fragment derived from digestion of the targeted protein in the sample) and an equivalent number of transition for the corresponding isotopically-labeled synthetic peptide. The selection of optimized transitions for particular surrogate peptides may be performed in an automated or semi-automated fashion using Thermo Scientific Pinpoint software, which identifies and refines an optimized set of transitions based on the expected transition intensity (determined using fragmentation rules and/or previously acquired data) and potential interferences (arising for example, from co-eluting peptide fragments derived from the sample matrix or other surrogate peptide fragments). The process of optimizing a set of transitions for a targeted peptide fragment is discussed in greater detail in U.S. patent application Ser. No. 12/163,928 for “Optimizing Selection of SRM Transitions for Analysis of Biomolecules by Tandem Mass Spectrometry”, the disclosure of which is incorporated herein by reference.

Table I lists the biomarkers or targeted proteins discovered by the present applicants to be differentially present in blood serum samples collected from ischemic and hemorrhagic stroke patients. The target proteins listed in the table consist of apolipoprotein A-I preprotein, apolipoprotein A-II preprotein, apolipoprotein A-IV preprotein, apolipoprotein B precursor, apolipoprotein C-I precursor, apolipoprotein C-II precursor, apolipoprotein C-III precursor, apolipoprotein D precursor, apolipoprotein E precursor and apolipoprotein H precursor. The table also lists, for each target protein, a surrogate peptide fragment produced by tryptic digestion, and optimized precursor-to-product ion transitions which were monitored by the triple quadrupole mass spectrometer for detection and quantitation of the surrogate peptide fragment, both in their wild-type and isotopically-labeled versions.

TABLE I

| Precursor Ion | Product Ion | | | |
|---|---|---|---|---|
| m/z | m/z | SEQID | Surrogate Peptide Sequence | Target Protein |
| 511.767 | 304.161 | 1 | ATVVYQGER | apolipoprotein H precursor |
| 511.767 | 326.656 | 1 | ATVVYQGER | apolipoprotein H precursor |
| 511.767 | 361.183 | 1 | ATVVYQGER | apolipoprotein H precursor |
| 511.767 | 376.19 | 1 | ATVVYQGER | apolipoprotein H precursor |
| 511.767 | 425.724 | 1 | ATVVYQGER | apolipoprotein H precursor |
| 511.767 | 489.241 | 1 | ATVVYQGER | apolipoprotein H precursor |
| 511.767 | 652.304 | 1 | ATVVYQGER | apolipoprotein H precursor |
| 511.767 | 751.373 | 1 | ATVVYQGER | apolipoprotein H precursor |
| 511.767 | 850.441 | 1 | ATVVYQGER | apolipoprotein H precursor |
| 516.771 | 314.169 | 1 | ATVVYQGER[HeavyR] | apolipoprotein H precursor |
| 516.771 | 331.66 | 1 | ATVVYQGER[HeavyR] | apolipoprotein H precursor |
| 516.771 | 371.191 | 1 | ATVVYQGER[HeavyR] | apolipoprotein H precursor |
| 516.771 | 381.194 | 1 | ATVVYQGER[HeavyR] | apolipoprotein H precursor |
| 516.771 | 430.728 | 1 | ATVVYQGER[HeavyR] | apolipoprotein H precursor |
| 516.771 | 499.249 | 1 | ATVVYQGER[HeavyR] | apolipoprotein H precursor |
| 516.771 | 662.313 | 1 | ATVVYQGER[HeavyR] | apolipoprotein H precursor |
| 516.771 | 761.381 | 1 | ATVVYQGER[HeavyR] | apolipoprotein H precursor |
| 516.771 | 860.45 | 1 | ATVVYQGER[HeavyR] | apolipoprotein H precursor |
| 647.338 | 262.139 | 2 | LKEFGNTLEDK | apolipoprotein C-I precursor |
| 647.338 | 388.693 | 2 | LKEFGNTLEDK | apolipoprotein C-I precursor |
| 647.338 | 504.266 | 2 | LKEFGNTLEDK | apolipoprotein C-I precursor |
| 647.338 | 590.796 | 2 | LKEFGNTLEDK | apolipoprotein C-I precursor |
| 647.338 | 605.314 | 2 | LKEFGNTLEDK | apolipoprotein C-I precursor |
| 647.338 | 719.356 | 2 | LKEFGNTLEDK | apolipoprotein C-I precursor |
| 647.338 | 776.378 | 2 | LKEFGNTLEDK | apolipoprotein C-I precursor |
| 647.338 | 923.446 | 2 | LKEFGNTLEDK | apolipoprotein C-I precursor |
| 647.338 | 1052.489 | 2 | LKEFGNTLEDK | apolipoprotein C-I precursor |
| 655.352 | 270.153 | 2 | LK[HeavyK]EFGNTLEDK[HeavyK] | apolipoprotein C-I precursor |
| 655.352 | 392.7 | 2 | LK[HeavyK]EFGNTLEDK[HeavyK] | apolipoprotein C-I precursor |
| 655.352 | 512.28 | 2 | LK[HeavyK]EFGNTLEDK[HeavyK] | apolipoprotein C-I precursor |
| 655.352 | 598.81 | 2 | LK[HeavyK]EFGNTLEDK[HeavyK] | apolipoprotein C-I precursor |
| 655.352 | 613.328 | 2 | LK[HeavyK]EFGNTLEDK[HeavyK] | apolipoprotein C-I precursor |
| 655.352 | 727.371 | 2 | LK[HeavyK]EFGNTLEDK[HeavyK] | apolipoprotein C-I precursor |
| 655.352 | 784.392 | 2 | LK[HeavyK]EFGNTLEDK[HeavyK] | apolipoprotein C-I precursor |
| 655.352 | 931.461 | 2 | LK[HeavyK]EFGNTLEDK[HeavyK] | apolipoprotein C-I precursor |
| 655.352 | 1060.503 | 2 | LK[HeavyK]EFGNTLEDK[HeavyK] | apolipoprotein C-I precursor |
| 643.799 | 305.181 | 3 | ESLSSYWESAK | apolipoprotein C-II precursor |
| 643.799 | 434.224 | 3 | ESLSSYWESAK | apolipoprotein C-II precursor |
| 643.799 | 435.703 | 3 | ESLSSYWESAK | apolipoprotein C-II precursor |
| 643.799 | 535.761 | 3 | ESLSSYWESAK | apolipoprotein C-II precursor |
| 643.799 | 620.303 | 3 | ESLSSYWESAK | apolipoprotein C-II precursor |
| 643.799 | 783.367 | 3 | ESLSSYWESAK | apolipoprotein C-II precursor |
| 643.799 | 870.399 | 3 | ESLSSYWESAK | apolipoprotein C-II precursor |
| 643.799 | 957.431 | 3 | ESLSSYWESAK | apolipoprotein C-II precursor |
| 643.799 | 1070.515 | 3 | ESLSSYWESAK | apolipoprotein C-II precursor |
| 647.806 | 313.196 | 3 | ESLSSYWESAK[HeavyK] | apolipoprotein C-II precursor |
| 647.806 | 439.71 | 3 | ESLSSYWESAK[HeavyK] | apolipoprotein C-II precursor |
| 647.806 | 442.238 | 3 | ESLSSYWESAK[HeavyK] | apolipoprotein C-II precursor |
| 647.806 | 539.768 | 3 | ESLSSYWESAK[HeavyK] | apolipoprotein C-II precursor |
| 647.806 | 628.318 | 3 | ESLSSYWESAK[HeavyK] | apolipoprotein C-II precursor |
| 647.806 | 791.381 | 3 | ESLSSYWESAK[HeavyK] | apolipoprotein C-II precursor |
| 647.806 | 878.413 | 3 | ESLSSYWESAK[HeavyK] | apolipoprotein C-II precursor |
| 647.806 | 965.445 | 3 | ESLSSYWESAK[HeavyK] | apolipoprotein C-II precursor |
| 647.806 | 1078.529 | 3 | ESLSSYWESAK[HeavyK] | apolipoprotein C-II precursor |
| 615.838 | 246.181 | 4 | NILTSNNIDVK | apolipoprotein D precursor |
| 615.838 | 361.208 | 4 | NILTSNNIDVK | apolipoprotein D precursor |
| 615.838 | 474.292 | 4 | NILTSNNIDVK | apolipoprotein D precursor |
| 615.838 | 502.274 | 4 | NILTSNNIDVK | apolipoprotein D precursor |
| 615.838 | 588.335 | 4 | NILTSNNIDVK | apolipoprotein D precursor |
| 615.838 | 702.378 | 4 | NILTSNNIDVK | apolipoprotein D precursor |
| 615.838 | 789.41 | 4 | NILTSNNIDVK | apolipoprotein D precursor |
| 615.838 | 890.457 | 4 | NILTSNNIDVK | apolipoprotein D precursor |
| 615.838 | 1003.541 | 4 | NILTSNNIDVK | apolipoprotein D precursor |
| 619.845 | 254.195 | 4 | NILTSNNIDVK[HeavyK] | apolipoprotein D precursor |
| 619.845 | 369.222 | 4 | NILTSNNIDVK[HeavyK] | apolipoprotein D precursor |
| 619.845 | 482.306 | 4 | NILTSNNIDVK[HeavyK] | apolipoprotein D precursor |
| 619.845 | 506.281 | 4 | NILTSNNIDVK[HeavyK] | apolipoprotein D precursor |
| 619.845 | 596.349 | 4 | NILTSNNIDVK[HeavyK] | apolipoprotein D precursor |
| 619.845 | 710.392 | 4 | NILTSNNIDVK[HeavyK] | apolipoprotein D precursor |
| 619.845 | 797.424 | 4 | NILTSNNIDVK[HeavyK] | apolipoprotein D precursor |
| 619.845 | 898.471 | 4 | NILTSNNIDVK[HeavyK] | apolipoprotein D precursor |
| 619.845 | 1011.555 | 4 | NILTSNNIDVK[HeavyK] | apolipoprotein D precursor |
| 578.85 | 260.196 | 5 | SKEQLTPLIK | apolipoprotein A-II preproprotein |
| 578.85 | 286.194 | 5 | SKEQLTPLIK | apolipoprotein A-II preproprotein |
| 578.85 | 373.28 | 5 | SKEQLTPLIK | apolipoprotein A-II preproprotein |
| 578 85 | 470.333 | 5 | SKEQLTPLIK | apolipoprotein A-II preproprotein |
| 578.85 | 471.287 | 5 | SKEQLTPLIK | apolipoprotein A-II preproprotein |
| 578.85 | 535.334 | 5 | SKEQLTPLIK | apolipoprotein A-II preproprotein |
| 578.85 | 684.465 | 5 | SKEQLTPLIK | apolipoprotein A-II preproprotein |
| 578.85 | 812.523 | 5 | SKEQLTPLIK | apolipoprotein A-II preproprotein |
| 578.85 | 941.566 | 5 | SKEQLTPLIK | apolipoprotein A-II preproprotein |
| 586.865 | 268.211 | 5 | SK[HeavyK]EQLTPLIK[HeavyK] | apolipoprotein A-II preproprotein |
| 586.865 | 290.201 | 5 | SK[HeavyK]EQLTPLIK[HeavyK] | apolipoprotein A-II preproprotein |
| 586.865 | 381.295 | 5 | SK[HeavyK]EQLTPLIK[HeavyK] | apolipoprotein A-II preproprotein |
| 586.865 | 475.294 | 5 | SK[HeavyK]EQLTPLIK[HeavyK] | apolipoprotein A-II preproprotein |
| 586.865 | 478.347 | 5 | SK[HeavyK]EQLTPLIK[HeavyK] | apolipoprotein A-II preproprotein |
| 586.865 | 543.348 | 5 | SK[HeavyK]EQLTPLIK[HeavyK] | apolipoprotein A-II preproprotein |
| 586.865 | 692.479 | 5 | SK[HeavyK]EQLTPLIK[HeavyK] | apolipoprotein A-II preproprotein |
| 586.865 | 820.538 | 5 | SK[HeavyK]EQLTPLIK[HeavyK] | apolipoprotein A-II preproprotein |
| 586.865 | 949.58 | 5 | SK[HeavyK]EQLTPLIK[HeavyK] | apolipoprotein A-II preproprotein |
| 450.224 | 226.137 | 6 | FWDYLR | apolipoprotein E precursor |
| 450.224 | 288.203 | 6 | FWDYLR | apolipoprotein E precursor |
| 450.224 | 376.69 | 6 | FWDYLR | apolipoprotein E precursor |
| 450.224 | 451.266 | 6 | FWDYLR | apolipoprotein E precursor |
| 450.224 | 566.293 | 6 | FWDYLR | apolipoprotein E precursor |
| 450.224 | 752.372 | 6 | FWDYLR | apolipoprotein E precursor |
| 455.228 | 231.141 | 6 | FWDYLR[HeavyR] | apolipoprotein E precursor |
| 455.228 | 298.211 | 6 | FWDYLR[HeavyR] | apolipoprotein E precursor |
| 455.228 | 381.694 | 6 | FWDYLR[HeavyR] | apolipoprotein E precursor |
| 455.228 | 461.274 | 6 | FWDYLR[HeavyR] | apolipoprotein E precursor |
| 455.228 | 576.301 | 6 | FWDYLR[HeavyR] | apolipoprotein E precursor |
| 455.228 | 762.38 | 6 | FWDYLR[HeavyR] | apolipoprotein E precursor |
| 602.644 | 465.756 | 7 | LGPHAGDVEGHLSFLEK | apolipoprotein A-IV precursor |
| 602.644 | 536.307 | 7 | LGPHAGDVEGHLSFLEK | apolipoprotein A-IV precursor |
| 602.644 | 545.942 | 7 | LGPHAGDVEGHLSFLEK | apolipoprotein A-IV precursor |
| 602.644 | 564.949 | 7 | LGPHAGDVEGHLSFLEK | apolipoprotein A-IV precursor |
| 602.644 | 579.811 | 7 | LGPHAGDVEGHLSFLEK | apolipoprotein A-IV precursor |
| 602.644 | 623.339 | 7 | LGPHAGDVEGHLSFLEK | apolipoprotein A-IV precursor |
| 602.644 | 701.354 | 7 | LGPHAGDVEGHLSFLEK | apolipoprotein A-IV precursor |
| 602.644 | 736.423 | 7 | LGPHAGDVEGHLSFLEK | apolipoprotein A-IV precursor |
| 602.644 | 769.883 | 7 | LGPHAGDVEGHLSFLEK | apolipoprotein A-IV precursor |
| 605.316 | 469.763 | 7 | LGPHAGDVEGHLSFLEK[HeavyK] | apolipoprotein A-IV precursor |
| 605.316 | 544.322 | 7 | LGPHAGDVEGHLSFLEK[HeavyK] | apolipoprotein A-IV precursor |
| 605.316 | 548.614 | 7 | LGPHAGDVEGHLSFLEK[HeavyK] | apolipoprotein A-IV precursor |
| 605.316 | 567.621 | 7 | LGPHAGDVEGHLSFLEK[HeavyK] | apolipoprotein A-IV precursor |
| 605.316 | 583.818 | 7 | LGPHAGDVEGHLSFLEK[HeavyK] | apolipoprotein A-IV precursor |
| 605.316 | 631.354 | 7 | LGPHAGDVEGHLSFLEK[HeavyK] | apolipoprotein A-IV precursor |
| 605.316 | 705.361 | 7 | LGPHAGDVEGHLSFLEK[HeavyK] | apolipoprotein A-IV precursor |
| 605.316 | 744.438 | 7 | LGPHAGDVEGHLSFLEK[HeavyK] | apolipoprotein A-IV precursor |
| 605.316 | 773.89 | 7 | LGPHAGDVEGHLSFLEK[HeavyK] | apolipoprotein A-IV precursor |
| 598.801 | 260.196 | 8 | GWVTDGFSSLK | apolipoprotein C-III precursor |
| 598.801 | 347.228 | 8 | GWVTDGFSSLK | apolipoprotein C-III precursor |
| 598.801 | 434.26 | 8 | GWVTDGFSSLK | apolipoprotein C-III precursor |
| 598.801 | 477.25 | 8 | GWVTDGFSSLK | apolipoprotein C-III precursor |
| 598.801 | 581.329 | 8 | GWVTDGFSSLK | apolipoprotein C-III precursor |
| 598.801 | 638.35 | 8 | GWVTDGFSSLK | apolipoprotein C-III precursor |
| 598.801 | 753.377 | 8 | GWVTDGFSSLK | apolipoprotein C-III precursor |
| 598.801 | 854.425 | 8 | GWVTDGFSSLK | apolipoprotein C-III precursor |
| 598.801 | 953.493 | 8 | GWVTDGFSSLK | apolipoprotein C-III precursor |
| 602.808 | 268.211 | 8 | GWVTDGFSSLK[HeavyK] | apolipoprotein C-III precursor |
| 602.808 | 355.243 | 8 | GWVTDGFSSLK[HeavyK] | apolipoprotein C-III precursor |
| 602.808 | 442.275 | 8 | GWVTDGFSSLK[HeavyK] | apolipoprotein C-III precursor |
| 602.808 | 481.257 | 8 | GWVTDGFSSLK[HeavyK] | apolipoprotein C-III precursor |
| 602.808 | 589.343 | 8 | GWVTDGFSSLK[HeavyK] | apolipoprotein C-III precursor |
| 602.808 | 646.364 | 8 | GWVTDGFSSLK[HeavyK] | apolipoprotein C-III precursor |
| 602.808 | 761.391 | 8 | GWVTDGFSSLK[HeavyK] | apolipoprotein C-III precursor |
| 602.808 | 862.439 | 8 | GWVTDGFSSLK[HeavyK] | apolipoprotein C-III precursor |
| 602.808 | 961.508 | 8 | GWVTDGFSSLK[HeavyK] | apolipoprotein C-III precursor |
| 806.896 | 482.26 | 9 | LLDNWDSVTSTFSK | apolipoprotein A-I preproprotein |
| 806.896 | 569.292 | 9 | LLDNWDSVTSTFSK | apolipoprotein A-I preproprotein |
| 806.896 | 670.34 | 9 | LLDNWDSVTSTFSK | apolipoprotein A-I preproprotein |
| 806.896 | 769.409 | 9 | LLDNWDSVTSTFSK | apolipoprotein A-I preproprotein |
| 806.896 | 856.441 | 9 | LLDNWDSVTSTFSK | apolipoprotein A-I preproprotein |
| 806.896 | 971.468 | 9 | LLDNWDSVTSTFSK | apolipoprotein A-I preproprotein |
| 806.896 | 1157.547 | 9 | LLDNWDSVTSTFSK | apolipoprotein A-I preproprotein |
| 806.896 | 1271.59 | 9 | LLDNWDSVTSTFSK | apolipoprotein A-I preproprotein |
| 806.896 | 1386.617 | 9 | LLDNWDSVTSTFSK | apolipoprotein A-I preproprotein |
| 810.903 | 490.275 | 9 | LLDNWDSVTSTFSK[HeavyK] | apolipoprotein A-I preproprotein |
| 810.903 | 577.307 | 9 | LLDNWDSVTSTFSK[HeavyK] | apolipoprotein A-I preproprotein |
| 810.903 | 678.354 | 9 | LLDNWDSVTSTFSK[HeavyK] | apolipoprotein A-I preproprotein |
| 810.903 | 777.423 | 9 | LLDNWDSVTSTFSK[HeavyK] | apolipoprotein A-I preproprotein |
| 810.903 | 864.455 | 9 | LLDNWDSVTSTFSK[HeavyK] | apolipoprotein A-I preproprotein |
| 810.903 | 979.482 | 9 | LLDNWDSVTSTFSK[HeavyK] | apolipoprotein A-I preproprotein |
| 810.903 | 1165.561 | 9 | LLDNWDSVTSTFSK[HeavyK] | apolipoprotein A-I preproprotein |
| 810.903 | 1279.604 | 9 | LLDNWDSVTSTFSK[HeavyK] | apolipoprotein A-I preproprotein |
| 810.903 | 1394.631 | 9 | LLDNWDSVTSTFSK[HeavyK] | apolipoprotein A-I preproprotein |
| 506.824 | 260.196 | 10 | TGISPLALIK | apolipoprotein B precursor |
| 506.824 | 327.731 | 10 | TGISPLALIK | apolipoprotein B precursor |
| 506.824 | 373.28 | 10 | TGISPLALIK | apolipoprotein B precursor |
| 506.824 | 427.789 | 10 | TGISPLALIK | apolipoprotein B precursor |
| 506.824 | 444.318 | 10 | TGISPLALIK | apolipoprotein B precursor |
| 506.824 | 654.454 | 10 | TGISPLALIK | apolipoprotein B precursor |
| 506.824 | 741.486 | 10 | TGISPLALIK | apolipoprotein B precursor |
| 506.824 | 854.57 | 10 | TGISPLALIK | apolipoprotein B precursor |
| 506.824 | 911.592 | 10 | TGISPLALIK | apolipoprotein B precursor |
| 510.831 | 268.211 | 10 | TGISPLALIK[HeavyK] | apolipoprotein B precursor |
| 510.831 | 331.738 | 10 | TGISPLALIK[HeavyK] | apolipoprotein B precursor |
| 510.831 | 381.295 | 10 | TGISPLALIK[HeavyK] | apolipoprotein B precursor |
| 510.831 | 431.796 | 10 | TGISPLALIK[HeavyK] | apolipoprotein B precursor |
| 510.831 | 452.332 | 10 | TGISPLALIK[HeavyK] | apolipoprotein B precursor |
| 510.831 | 662.469 | 10 | TGISPLALIK[HeavyK] | apolipoprotein B precursor |
| 510.831 | 749.501 | 10 | TGISPLALIK[HeavyK] | apolipoprotein B precursor |
| 510.831 | 862.585 | 10 | TGISPLALIK[HeavyK] | apolipoprotein B precursor |
| 510.831 | 919.606 | 10 | TGISPLALIK[HeavyK] | apolipoprotein B precursor |

Once the mass spectrometric analysis of the prepared sample has been completed, the quantities of the surrogate peptides in the sample may be determined by integration of the relevant peak areas, as known in the prior art, step 170. When isotopically-labeled internal standards are used, as described above, the quantities of the surrogate peptides of interest are established via an empirically-derived or predicted relationship between surrogate peptide quantity (which may be expressed as concentration) and the area ratio of the surrogate peptide and internal standard peaks at specified transitions. Other implementations of the assay may utilize external standards or other expedients for peptide quantification. It will be recognized that the mass spectrometry-based assay described herein advantageously enables the concurrent measurement of multiple target proteins in a single test.

While the embodiment described above employs tandem mass spectrometry for measurement of the targeted protein, alternative embodiments of the invention may employ a mass spectrometer capable of accurate mass measurement (e.g., one having an Orbitrap or Fourier Transform/Ion Cyclotron Resonance mass analyzer) to uniquely identify and quantitate ions corresponding to the target protein(s) without requiring an ion fragmentation stage.

After the amount of the target protein in the sample have been measured in accordance with the steps described above, the measured amount is compared to a control amount to assess whether the tested sample indicates an ischemic or hemorrhagic stroke, step 180. The control amount may represent an empirically-derived threshold or cutoff value that provides a desired degree of specificity and/or sensitivity. The threshold value may be calculated or selected by well-known statistical techniques, for example by construction and analysis of receiver operator characteristic (ROC) curves, as discussed below. Without limitation, the comparison step may involve comparison of the measured amount of a single target protein to a control amount, comparison of the measured amounts of a plurality of targeted proteins (otherwise referred to as a biomarker panel) to corresponding control amounts, or comparison of a ratio of the measured amounts of two target proteins to a control amount representative of a corresponding ratio. The results of the mass spectrometry assay may also be combined or considered in conjunction with other methods for differentiating between ischemic and hemorrhagic stroke (e.g., existing imaging-based techniques) in order to improve the sensitivity and/or selectivity of diagnosis.

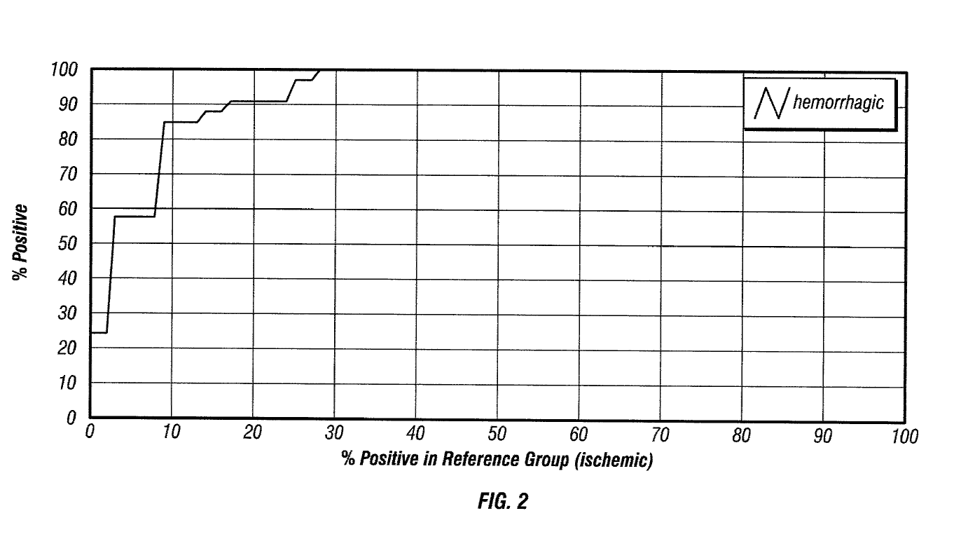

FIG. 2 presents an ROC curve depicting the sensitivity-specificity relationship for apolipoprotein A-IV preprotein as a biomarker for the differential determination of ischemic and hemorrhagic stroke. This protein was found by the applicants to have the greatest discriminatory power as a single biomarker. As known in the art, the area under an ROC curve (AUC) is indicative of the measure of a diagnostic assay's discriminatory power, with an AUC value of 1.0 representing a “perfect” assay (i.e., 100% sensitive and 100% selective), and an AUC value of 0.5 representing no discriminative value. The ROC curve was derived from analysis of samples obtained on the day of stroke from a patient population of twenty individuals definitively diagnosed (by traditional methods) with either ischemic or hemorrhagic stroke. This ROC curve has an AUC of approximately 0.9, indicating that measurement of the apolipoprotein A-IV preprotein has very good discriminatory power as a test for the determination of hemorrhagic versus ischemic stroke. It may be discerned from the ROC curve that a determination of hemorrhagic as opposed to ischemic stroke may be achieved based on the measured level of apolipoprotein A-IV preprotein at a sensitivity of about 85% with a selectivity of >90%.

As noted above, improved discriminatory power may be achieved by employing testing of multiple targeted proteins, or biomarker panels. Table II lists ROC AUC's determined (from analysis of the day-of-stroke samples mentioned above) for various biomarker panels, where the measured amounts of the targeted proteins are considered either in combination or in ratio form. It is noted that several combinations of biomarkers, for example, apolipoprotein A-IV precursor and apolipoprotein C-II precursor, and apolipoprotein B precursor and apolipoprotein A-II preproprotein, yield AUC's of 1, indicative of perfect discriminatory'power.

TABLE II

| Biomarker 1 | And/ratio | Biomarker 2 | AUC |
|---|---|---|---|
| apolipoprotein A-IV precursor | and | apolipoprotein C-II precursor | 1 |
| apolipoprotein B precursor | and | apolipoprotein A-II preproprotein | 1 |
| apolipoprotein H precursor | and | apolipoprotein C-II precursor | 1 |
| apolipoprotein A-I preproprotein | and | apolipoprotein A-II preproprotein | 1 |
| apolipoprotein C-I precursor | and | apolipoprotein A-II preproprotein | 1 |
| apolipoprotein C-III precursor | and | apolipoprotein A-II preproprotein | 1 |
| apolipoprotein E precursor | and | apolipoprotein A-II preproprotein | 0.997 |
| apolipoprotein H precursor | and | apolipoprotein B precursor | 0.994 |
| apolipoprotein B precursor | and | apolipoprotein A-I preproprotein | 0.988 |
| apolipoprotein C-III precursor | and | apolipoprotein A-IV precursor | 0.985 |
| apolipoprotein E precursor | and | apolipoprotein A-IV precursor | 0.979 |
| apolipoprotein D precursor | and | apolipoprotein A-IV precursor | 0.976 |
| apolipoprotein C-I precursor | and | apolipoprotein A-IV precursor | 0.973 |
| apolipoprotein D precursor | and | apolipoprotein A-IV precursor | 0.973 |
| apolipoprotein H precursor | and | apolipoprotein A-I preproprotein | 0.973 |
| apolipoprotein E precursor | and | apolipoprotein A-I preproprotein | 0.967 |
| apolipoprotein A-II preproprotein | and | apolipoprotein A-IV precursor | 0.964 |
| apolipoprotein C-III precursor | and | apolipoprotein D precursor | 0.933 |
| apolipoprotein B precursor | and | apolipoprotein C-II precursor | 0.921 |
| apolipoprotein H precursor | and | apolipoprotein A-II preproprotein | 0.912 |
| apolipoprotein A-I preproprotein | and | apolipoprotein A-IV precursor | 0.903 |
| apolipoprotein H precursor | and | apolipoprotein A-IV precursor | 0.897 |
| apolipoprotein A-I preproprotein | and | apolipoprotein D precursor | 0.894 |
| apolipoprotein B precursor | and | apolipoprotein A-IV precursor | 0.891 |
| apolipoprotein C-III precursor | and | apolipoprotein C-II precursor | 0.882 |
| apolipoprotein B precursor | and | apolipoprotein E precursor | 0.879 |
| apolipoprotein B precursor | and | apolipoprotein C-III precursor | 0.873 |
| apolipoprotein B precursor | ratio | apolipoprotein A-IV precursor | 0.873 |
| apolipoprotein A-IV precursor | ratio | apolipoprotein C-II precursor | 0.867 |
| apolipoprotein C-I precursor | and | apolipoprotein D precursor | 0.861 |

The following example is offered by way of illustration and should not be construed as limiting the invention, the scope of which should be determined with reference to the appended claims.

### EXAMPLE

### Serum Processing

Serum samples were prepared by diluting serum 1:4 v/v with 8M GuHCl/150 mM Tris/10 mM DTT pH 8.5. Samples were incubated at 37° C. for 60 minutes and then cooled to room temperature. Next, 500 mM iodoacetic acid/1M Tris pH 8.5 was added to each sample to a final concentration of 40 mM. Samples were alkylated in the dark at room temperature for 60 minutes. The reaction was quenched with the addition of 2 M OTT to a final concentration of 5 mM. Post quench, samples were diluted to 1 ml with the addition of 50 mM Tris/5 mM CaCl₂, pH 8.0 and 10 μg of sequencing grade trypsin (Promega) were added to each sample prior to incubation at 37° C. for 24 hours. The digestion reaction was quenched with the addition of TFA to 1%. Subsequent to digestion, the samples were processed by solid phase extraction for desalting and clean-up using Hypersep™ C18 50 mg 96 well plates (Thermo Fisher Scientific). Plates were equilibrated once with 100% ACN and then rinsed twice with 0.25% TFA. Samples were then loaded and wells were rinsed 5 times with 0.25% TFA. The peptides were eluted with 400 μl of 75% ACN/0.1% formic acid at 300 rcf in a centrifuge equipped with a swinging bucket rotor. After elution, the samples were lyophilized. Just before loading on the mass spectrometer, peptides were resuspended in 97% H₂O/3% ACN/0.25% formic acid.

### Selective Reaction Monitoring (SRM) Assays

SRM assays were developed on a Vantage triple quadrupole mass spectrometer, Surveyor MS pump, CTC PAL Autosampler and an IonMax Source equipped with a high flow metal needle (Thermo Fisher Scientific). Reverse phase separations were carried out on a 1 mm×50 mm Hypersil Gold 1.9 μm C18 particle (Thermo Fisher Scientific). Solvent A was LC-MS grade water with 0.2% (v/v) formic acid, and solvent B was LC-MS grade 30% (v/v) acetonitrile with 0.2% (v/v) formic acid (Optima grade reagents, Thermo Fisher Scientific). Pinpoint software (Thermo Fisher Scientific) was used for targeted protein quantitation. The recently developed software algorithm automates the prediction of candidate peptides and the choice of multiple fragment ions for SRM assay design. In addition, Pinpoint creates the instrument method and sequence file, and automates peptide identity confirmation and quantitative data processing. For the workflow described herein, the differentially expressed peptides identified in the LC-MS/MS discovery MS data were imported directly into Pinpoint and transitions were chosen based upon the predominant fragments observed in the discovery data (>5 transitions per peptide). Ultimately, we chose one proteotypic peptide and several transitions per target protein in order to simplify the assay. Peptides were identified by the co-eluting light and heavy-labeled transitions in the chromatographic separation. For additional verification, the transition ratios were confirmed using discovery spectra. Time alignment and relative quantification of the transitions was performed with Pinpoint. All clinical samples were assayed in triplicate.

### ROC Analysis

When considering the outcome of a specific measurement between disease and normal populations, typically there is some degree of overlap and the two populations are not perfectly separated. In a traditional ROC analysis, calculating the true positive (TP) rate (sensitivity) and the false positive (FP) rate (100-specificity) is one way of assessing the efficacy of the specific measurement to differentiate the two populations. In this case, accounting TPs and FPs for the measurement while sweeping the cut-off point or threshold used to discriminate the two populations is used to construct a ROC curve.

### RESULTS

Using the techniques described above, the ratios of expression levels of 9 peptide fragments representing 9 target apolipoproteins were shown to be differentially expressed in plasma or serum samples from patients who had strokes relative to patients who did not have strokes.

What is claimed is:

1. A method of differentiating between ischemic stroke and hemorrhagic stroke in a human patient, comprising:
obtaining a biological sample from the patient;
analyzing the biological sample by mass spectrometry to measure the amount of at least one targeted protein differentially present in ischemic stroke and hemorrhagic stroke patients, the at least one targeted protein being selected from a group consisting of apolipoprotein A-I preprotein, apolipoprotein A-II preprotein, apolipoprotein A-IV preprotein, apolipoprotein B precursor, apolipoprotein C-I precursor, apolipoprotein C-II precursor, apolipoprotein C-III precursor, apolipoprotein D precursor, apolipoprotein E precursor and apolipoprotein H precursor; and
comparing the measured amount to a control amount.

2. The method of claim 1, wherein the at least one targeted protein includes apolipoprotein A-IV preprotein.

3. The method of claim 2, wherein the at least one targeted protein further includes at least one of apolipoprotein C-I precursor, apolipoprotein C-II precursor, apolipoprotein C-III precursor and apolipoprotein D precursor.

4. The method of claim 3, wherein the step of comparing the measured amount to the control level includes calculating a ratio of the measured amount of apolipoprotein A-IV preprotein to the measured amount of another targeted protein.

5. The method of claim 4, wherein the other targeted protein is apolipoprotein C-II precursor.

6. The method of claim 1, wherein the step of analyzing the biological sample includes:
enzymatically digesting the sample to produce peptide fragments; and
measuring the amount of at least one peptide fragment produced by the at least one targeted protein.

7. The method of claim 6, wherein the step of analyzing the biological sample includes performing selective reaction monitoring (SRM) of ions generated from the sample in a triple quadrupole mass spectrometer.

8. The method of claim 7, wherein the at least one targeted protein includes apolipoprotein A-IV preprotein, and the step of analyzing the biological sample includes measuring the amount of SEQ ID NO. 7 (LGPHAGDVEGHLSFLEK).

9. The method of claim 8, wherein the step of measuring the amount of LGPHAGDVEGHLSFLEK includes monitoring at least one precursor-to-product ion transition selected from a group consisting of 603→466, 603→536, 603→546, 603→565, 603→580, 603→623, 603→701, 603→736 and 603→770.

10. The method of claim 7, wherein the at least one targeted protein includes apolipoprotein H precursor, and the step of analyzing the biological sample includes measuring the amount of SEQ ID NO. 1 (ATVVYQGER).

11. The method of claim 7, wherein the at least one targeted protein includes apolipoprotein C-I precursor, and the step of analyzing the biological sample includes measuring the amount of SEQ ID NO. 2 (LKEFGNTLEDK).

12. The method of claim 7, wherein the at least one targeted protein includes apolipoprotein C-II precursor, and the step of analyzing the biological sample includes measuring the amount of SEQ ID NO. 3 (ESLSSYWESAK).

13. The method of claim 7, wherein the at least one targeted protein includes apolipoprotein D precursor, and the step of analyzing the biological sample includes measuring the amount of SEQ ID NO. 4 (NILTNNIDVK).

14. The method of claim 7, wherein the at least one targeted protein includes apolipoprotein A-II preprotein, and the step of analyzing the biological sample includes measuring the amount of SEQ ID NO. 5 (SKEQLTPLIK).

15. The method of claim 7, wherein the at least one targeted protein includes apolipoprotein E precursor, and the step of analyzing the biological sample includes measuring the amount of SEQ ID NO. 6 (FWDYLR).

16. The method of claim 7, wherein the at least one targeted protein includes apolipoprotein C-IIII precursor, and the step of analyzing the biological sample includes measuring the amount of SEQ ID NO. 8 (GWVTDGFSSLK).

17. The method of claim 7, wherein the at least one targeted protein includes apolipoprotein A-I preprotein, and the step of analyzing the biological sample includes measuring the amount of SEQ ID NO. 9 (LLDNWDSVTSTFSK).

18. The method of claim 7, wherein the at least one targeted protein includes apolipoprotein B precursor, and the step of analyzing the biological sample includes measuring the amount of SEQ ID NO. 10 (TGISPLALIK).

19. The method of claim 1, wherein the step of comparing the measured amount to the control amount includes calculating a ratio of the measured amount of a first targeted protein to the measured amount of a second protein targeted protein.

20. The method of claim 1, wherein the step of analyzing the biological sample includes separating components of the biological sample by liquid chromatography prior to mass spectrometric analysis.

21. The method of claim 1, wherein the step of analyzing the biological sample includes purifying or concentrating components of the sample prior to mass spectrometric analysis.

22. The method of claim 1, wherein the at least one targeted protein includes a plurality of targeted proteins.

23. The method of claim 22, wherein the plurality of targeted proteins includes at least three targeted proteins.

24. The method of claim 1, wherein the biological fluid is blood serum.


SEQUENCE LISTING (10 sequences)

SEQ ID NO: 1 - protein, 9 aa, Homo sapiens
ATVVYQGER

SEQ ID NO: 2 - protein, 11 aa, Homo sapiens
LKEFGNTLEDK

SEQ ID NO: 3 - protein, 11 aa, Homo sapiens
ESLSSYWESAK

SEQ ID NO: 4 - protein, 11 aa, Homo sapiens
NILTSNNIDVK

SEQ ID NO: 5 - protein, 10 aa, Homo sapiens
SKEQLTPLIK

SEQ ID NO: 6 - protein, 6 aa, Homo sapiens
FWDYLR

SEQ ID NO: 7 - protein, 17 aa, Homo sapiens
LGPHAGDVEGHLSFLEK

SEQ ID NO: 8 - protein, 11 aa, Homo sapiens
GWVTDGFSSLK

SEQ ID NO: 9 - protein, 14 aa, Homo sapiens
LLDNWDSVTSTFSK

SEQ ID NO: 10 - protein, 10 aa, Homo sapiens
TGISPLALIK